(19) United States
(12) Patent Application Publication
(10) Pub. No.: US 2002/0015982 A1
(43) Pub. Date: Feb. 7, 2002

(54) METHOD OF PURIFYING DNA IN A CROSS-FLOW CENTRIFUGE

(75) Inventors: WOLFGANG KUHNE, PENZBERG (DE); FRIEDRICH POPP, PENZBERG (DE)

Correspondence Address: GEORGE W JOHNSTON, HOFFMANN LA ROCHE INC, 340 KINGSLAND STREET, NUTLEY, NJ, 07110

(73) Assignee: Roche Diagnostics GmbH

(21) Appl. No.: 09/341,367

(22) PCT Filed: Jan. 9, 1998
(86) PCT No.: PCT/EP98/00104

(30) Foreign Application Priority Data
Jan. 10, 1997 (EP) 97100330.6

(51) Int. Cl. C12Q 1/68; C07H 21/00; C07H 21/02; C07H 21/04; C12P 19/34

(52) U.S. Cl. 435/91.1; 435/6

(57) ABSTRACT

The invention concerns a method of purifying extrachromosomal DNA by passing an extrachromosomal DNA and fluid containing further cell components through a cross-flow centrifuge under given conditions, resulting in separation of the extrachromosomal DNA from the other cell components such that purified extrachromosomal DNA is obtained. The invention further concerns the use of the purified extrachromosomal DNA for cloning, transformation, transfection and microinjection into cells, for use in gene therapy processes, DNA vaccination and/or for polymerase chain reaction (PCR). The invention finally concerns the use of a cross-flow centrifuge for purifying extrachromosomal DNA.

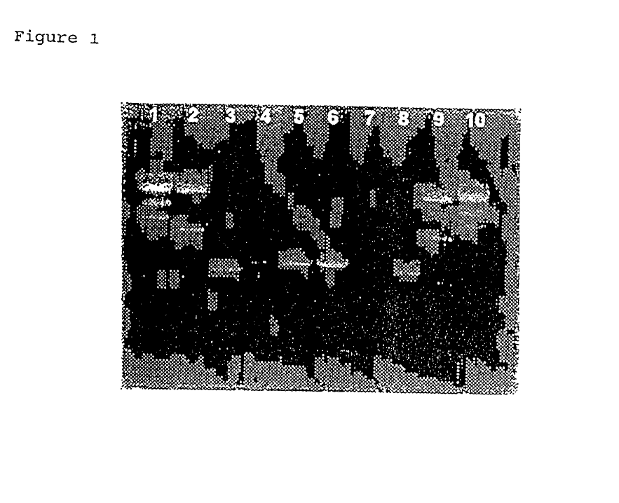

### DESCRIPTION

The present invention concerns a process for purifying extrachromosomal DNA using a continuous flow centrifuge.

The isolation of nucleic acids and in particular of plasmid DNA is of major importance in molecular biology and in modern medicine. Plasmid DNA refers to extrachromosomal DNA duplex molecules which usually have a size from 1 kb up to more than 200 kb and are present in host cells in one to several hundred copies. Plasmid DNA is usually amplified in cells e.g. in gram-negative bacteria, in particular in E. coli. Afterwards the cells are lysed and the plasmid DNA is isolated from them. The isolated plasmid DNA can then be used for molecular biological or medical applications e.g. to construct cloning vectors, to transform prokaryotic cells and to transfect eukaryotic cells. Various methods are known to lyse the cells and to isolate the plasmid DNA (see J. Sambrook et al., Molecular Cloning, A Laboratory Manual, 2nd edition, 1989, Cold Spring Harbor Laboratory Press).

In a process developed by Birnboim & Doly for the isolation of plasmid DNA from cells (Birnboim & Doly, Nucl. Acid Res. 7 (1979) 1513-1523) the biomass is lysed with an NaOH/detergent solution and subsequently the pH value is adjusted to ca. 5.0 with K acetate. A precipitate is formed in this process that mainly contains genomic DNA and cell wall fragments. In order to separate these impurities, the suspension is transferred into centrifuge buckets. The precipitate is then centrifuged with a bucket centrifuge in order to obtain the supernatant containing the plasmid DNA.

Centrifuges are also commonly used in fermentation processes in order for example to separate fermenter supernatants from cells and cell fragments. Screen centrifuges and fixed wall centrifuges are usually used for this (see Gerhartz W., Enzymes in industry: production and applications, 1990, VCH, Weinheim, Germany, chapter 3.2.1).

With common laboratory centrifuges the volume that can be processed is limited by the volume of the rotor and the rotor buckets to less than ten liters (e.g. Sorvall centrifuge, GSA rotor, 6×250 ml). Hence this process can only be used to isolate plasmid DNA in small amounts. An application of the method to large-scale processes is very problematic due to the limited centrifuge volume.

Large volumes can be processed by means of continuous flow centrifuges. WO92/12780 describes a technical design of a continuous flow centrifuge and its use for separating macromolecule mixtures. In this process four standard proteins are for example separated in an aqueous two phase system at a maximum of 1000 rpm depending on the respective distribution coefficients of the proteins. The components of the mixture are obtained separated from one another as a result of the differences in elution times.

However, the demand for purified plasmid DNA for analytical and therapeutic applications in research and medicine is increasing due to the expanding use of molecular biological methods. Hence an object of the present invention was to provide a process which enables an efficient and rapid purification of plasmid DNA in large amounts.

A first aspect of the present invention concerns a process for the purification of extrachromosomal DNA which is characterized in that a liquid containing extrachromosomal DNA and other cell components is passed through a continuous flow centrifuge under conditions which lead to a separation of the extrachromosomal DNA from insoluble cell components and the purified extrachromosomal DNA is isolated.

In the prior art continuous flow centrifuges have previously been used only for cell separation. It was now surprisingly found that continuous flow centrifuges can also be used to purify extrachromosomal DNA in large amounts without damage to the extrachromosomal DNA by the resulting shear forces. It was also surprising that the chromosomal DNA present in the suspension of the lysed cells is not fragmented during the continuous flow centrifugation and can thus be separated quantitatively from the extrachromosomal DNA.

The extrachromosomal DNA which is purified by the method according to the invention can be linear or circular, single-stranded or double-stranded. The DNA is preferably a circular and double-stranded plasmid DNA. The cell containing the extrachromosomal DNA can be a prokaryotic or eukaryotic cell; it is preferably a bacterial cell and in particular a gram-negative cell such as an E. coli cell. Optionally cells can be used which contain so-called artificial chromosomes as extrachromosomal DNA. Artificial chromosomes are linear double-stranded DNA molecules which are generally named YAC (yeast artificial chromosome) and are amplified in yeast cells.

The liquid containing the extrachromosomal DNA that is used in the process according to the invention is preferably a cell lysate. The cell lysate is particularly preferably prepared by alkaline lysis of cells containing extrachromosomal DNA and subsequent acidification. However, it is also possible to use other common methods of cell lysis such as a combination of enzyme (lysozyme) and heat treatment.

Any desired amount of cellular biomass can be used as a starting material for the process according to the invention. A biomass of 100 g to 50 kg is preferably lysed per batch.

The liquid containing extrachromosomal DNA is usually passed into the continuous flow centrifuge by a gradient or/and pumps. In the process according to the invention a continuous flow centrifuge is used with a volume adapted to the lysis preparation. A volume of at least 0.1 to 50 l is preferably used and a volume of 0.2 to 4 l is particularly preferably used. The centrifuge container is preferably cylindrical. The continuous flow centrifuge is operated at a suitable g number, preferably at 10,000 to 40,000×g. Examples of commercially available continuous flow centrifuges are CEPA rapid centrifuges or high performance centrifuges from the Carr Co. (USA) which at present have a capacity of up to 9,000 l/h.

The process according to the invention is generally carried out continuously. The suspension of the lysed biomass is passed into the continuous flow centrifuge from below. As a result of the rotation of the centrifuge vessel (10,000-40,000×g) solid components such as cell wall components and genomic DNA attached thereto are deposited on the wall of the centrifuge vessel. The solution containing purified extrachromosomal DNA usually passes out of the top of the continuous flow centrifuge although it is also conceivable that the solution containing the extrachromosomal DNA flows out of the sides, the bottom or other positions.

The continuous flow centrifuge can be operated at different temperatures; the process is preferably carried out at 4° C. to room temperature.

In the present process it is possible to purify extrachromosomal DNA of different sizes; preferably extrachromosomal DNA with a size of 1 kbp to 200 kbp is purified. The extrachromosomal DNA is preferably linear, circular or supercoiled plasmid DNA.

After leaving the centrifuge the extrachromosomal DNA can be further purified. Hence an RNase treatment is optionally carried out in order to remove RNA from the solution. In addition it is also possible to carry out chromatographic purification steps such as anion exchange chromatography, affinity chromatography or hydroxylapatite chromatography. Examples of suitable materials for anion exchange chromatography are organic or inorganic polymers and copolymers such as polymethacrylate (Macroprep-Biorad, Germany), polystyrenedivinylbenzene (Poros-Perseptive, HyperD-Biosepra, Source Pharmacia) or silica gel on the surface of which positively-charged groups such as diethylaminoethyl (DEAE) or dimethylaminoethyl (DMAE) groups are bound. A particularly preferred material for anion exchange chromatography is Q-Sepharose. A particularly preferred material for affinity chromatography is hydroxylapatite.

In addition the DNA solution that is obtained can be subjected to a cross-flow filtration for additional purification, concentration or/and rebuffering. In this cross-flow filtration it is also possible to achieve a substantial removal of endotoxins from the DNA preparation. For this the DNA solution is guided tangentially past one or several semipermeable membranes whose exclusion size is chosen such that the DNA molecules are retained by the membranes and substances with a lower molecular weight can pass through the membranes to obtain an endotoxin-free DNA solution.

The extrachromosomal DNA obtained by the process according to the invention is essentially undamaged and has essentially no single-strand or double-strand breaks. In particular a plasmid DNA purified according to the invention exhibits only one dominant band after separation by gel electrophoresis which corresponds to the “covalently closed circle” conformation. Furthermore there are no other bands apart from the bands corresponding to the open circle and linearized circle conformations.

The DNA obtained by the process according to the invention can be used directly for standard molecular biological and medical applications such as for cloning, for transformation, for transfection, for microinjection into cells, for use in methods of gene therapy, DNA vaccination or/and for the polymerase chain reaction (PCR).

A further aspect of the present invention concerns the use of a continuous flow centrifuge for purifying extrachromosomal DNA.

### EXAMPLE

In the experiment a CEPA laboratory centrifuge LE (open design) with a clarifying cylinder made of stainless steel (1.4571, V4A) is used. Ca. 2000 g biomass is lysed by the alkaline lysis method (modified method according to Birnboim & Doly, Birnboim & Doly, Nucl.Acid Res. 7 (1979) 1513-1523).

1. Lysis of the E. coli biomass

2000 g wet E. coli biomass from the fermenter is filled into depyrogenized beakers. 22.5 l resuspension buffer (50 mmol/l Tris-HCl, 10 mmol/l EDTA-Na₂, pH 8±0.2) is added and slowly stirred (ca. 35 rpm) for at least 24 hours at 5±4° C. until the biomass is completely suspended. Then the temperature of the suspension is slowly increased to 25° C. 22.5 l 0.2 mol/l NaOH, 1% SDS is added to the suspension while stirring at ca. 80 rpm and incubated for 5 minutes at 25° C. 22.5 l potassium acetate buffer (3 mol/l potassium acetate buffer pH 5.5) is added while stirring and the temperature of the biomass is reduced as rapidly as possible to 4° C. The lysate that is obtained is clear filtered with the aid of a continuous flow centrifuge in the continuous flowthrough mode.

2. Continuous flow centrifugation

The viscous suspension is pumped into the continuous flow centrifuge through the inlet opening. During this the centrifuge is operated at a g number of 10,000-18,000×g. As soon as the liquid that flows out becomes turbid, the precipitate must be removed from the cylinder and the centrifugation is continued after inserting the cleaned cylinder. The clear plasmid DNA solution which has been freed of cellular impurities emerges from the top of the continuous flow centrifuge and is collected in a vessel.

3. Additional purification steps:

Q-Sepharose chromatography, hydroxylapatite chromatography and cross-flow filtration

In a next step a chromatography on Q-Sepharose and hydroxylapatite is carried out. The decanted centrifuge supernatant is adjusted to a conductivity of 49-50 mS/cm by addition of TE buffer (10 mmol/l Tris-HCl, 1 mmol/l EDTA pH 8.5±0.2) and cooled to 5±4° C. The entire chromatography is carried out at this temperature. The centrifugation supernatant is absorbed onto the equilibrated column. Subsequently the column is washed with ca. 8 CV 10 mmol/l Tris-HCl, 1 mmol/l EDTA, 0.65 mol/l NaCl pH 8.5±0.2.

For the elution a gradient (5 CV buffer A (10 mmol/l Tris-HCl, 1 mmol/l EDTA, 0.65 mmol/l NaCl, pH 8.0±2), 5 CV buffer B (10 mmol/l Tris-HCl, 1 mmol/l EDTA, 0.85 mol/l NaCl pH 8.0±0.2)) is applied to the column and the eluate is fractionated, the detection is carried out at 254 nm. The prepeak (impurities) is separated from the main peak (plasmid DNA) by collecting the main peak in a separate vessel starting from the ascending flank.

Subsequently a chromatography on hydroxylapatite (HA ceramic) is carried out at 5±4° C.

Equilibration buffer: 0.1 mol/l potassium phosphate, 6 mol/l urea pH 7.0±0.2.

Wash buffer 1: 0.15 mol/l potassium phosphate, 6 mol/l urea pH 7.0±0.2.

Wash buffer 2: 0.02 mol/l potassium phosphate buffer pH 7.0±0.2.

Elution buffer: 0.5 mol/l potassium phosphate pH 7.0±0.2.

The detection is carried out at 254 nm using a UV detector/recorder unit. A 1% product solution (plasmid DNA) is used as a calibration solution that was measured with a calibrated photometer.

The Q-Sepharose pool is adjusted to a final concentration of 1.1 mmol/l calcium chloride and absorbed onto the equilibrated column.

Then the column is successively washed with:

1. 0.1 mol/l potassium phosphate, 6 mol/l urea pH 7.0±0.2 until absorbance is no longer detectable at the detector.

2. 2-4 CV, 0.15 mol/l potassium phosphate, 6 mol/l urea pH 7.0±0.2

3. 5 CV, 0.02 mol/l potassium phosphate pH 7.0±0.2.

It is eluted with 0.5 mol/l potassium phosphate buffer pH 7.0±0.1 after the wash steps at a flow rate of 5-6 CV/h.

The peak is pooled and concentrated to ca. 50 ml with a cross-flow filtration. The CFF is carried out at a retentate flow rate of 100-200 l/h.m², a transmembrane pressure of Ca. 0.8 bar and an cross-flow pressure of Ca. 1.2 bar. The retentate is subsequently flow diafiltered against TE buffer (10 mmol/l Tris-HCl, 1 mmol/l EDTA, pH 8.0) until the values for pH and conductivity of the retentate and TE buffer agree. After completion of the diafiltration process the retentate is adjusted to a plasmid DNA concentration of 1 mg/ml by dilution with diafiltration buffer.

4. Gel electrophoresis

The intactness of the plasmid DNA that was obtained is checked by means of agarose gel electrophoresis.

For this an aliquot of the plasmid DNA is applied at various concentrations to an agarose gel. The illustrated agarose gel shows the DNA length standard No. II (fragment sizes: 125, 564, 2027, 2322, 4361, 6557, 9416, 23130 bp) in lanes 1 and 10 and the DNA length standard No. III (fragment sizes: 125, 564, 831, 947, 1375, 1584, 1904, 2027, 3530, 4268, 4973, 5148, 21226 bp) in lanes 2 and 9. pBR322 (4162 bp) is applied as a reference plasmid in lane 3 which was purified by a conventional caesium chloride gradient method. It is known that plasmid DNA purified by this method essentially contains plasmid DNA which corresponds to the covalently closed circle conformation (dominant supercoiled band). The plasmid DNA (pCMV-CAT) purified by the method according to the invention is applied in different amounts in lanes 4, 5 and 6.

This plasmid DNA was further purified after the process according to the invention by means of Q-Sepharose and hydroxylapatite chromatography and by cross-flow filtration.

Legend:

1% Agarose gel

Lane 1: DNA length standard II (Boehringer Mannheim GmbH; Cat. No. 236250)

Lane 2: DNA length standard III (Boehringer Mannheim GmbH, Cat. No. 528552).

Lane 3: pBR322 (Boehringer Mannheim GmbH, Cat. No. 481238) (0.4 μg)

Lane 4: pCMV-CAT after CFF, 0.19 μg (bulk active substance solution)

Lane 5: pCMV-CAT after CFF, 0.45 μg (bulk active substance solution)

Lane 6: pCMV-CAT after CFF, 0.71 μg (bulk active substance solution)

Lane 7: TE buffer

Lane 8: pBR322 (Boehringer Mannheim GmbH, Cat. No. 481238) (0.4 μg)

Lane 9: DNA length standard III (Boehringer Mannheim GmbH; Cat. No. 528552)

Lane 10: DNA length standard II (Boehringer Mannheim GmbH, Cat. No. 236250).

The plasmid DNA purified according to the invention, like the reference plasmid DNA (lane 3), essentially shows a dominant band. This shows that the plasmid DNA isolated according to the invention is not damaged and retains its original conformation. In addition the absence of additional bands in the agarose gel shows that the chromosomal DNA contained in the lysed cell suspension is not fragmented during the continuous flow centrifugation but can be completely separated as a precipitated macromolecule from the plasmid DNA.

1. Process for the purification of extrachromosomal DNA
wherein
a liquid, which is a cell lysate, containing extrachromosomal DNA and other cell components is passed through a continuous flow centrifuge operated at an acceleration of 10,000 to 40,000×g without prior centrifugation steps in a continuous process and conditions are adhered to which lead to separation of the extrachromosomal DNA from insoluble cell components and the purified extrachromosomal DNA is isolated.

2. Process as claimed in claim 1,
wherein
the lysis is an alkaline lysis.

3. Process as claimed in one of the previous claims,
wherein
the cell containing extrachromosomal DNA is a bacterial cell, preferably an E. coli cell.

4. Process as claimed in one of the previous claims,
wherein
the liquid passed into the centrifuge is obtained by lysing 100 g-50 kg biomass.

5. Process as claimed in one of the previous claims,
wherein
the liquid containing extrachromosomal DNA is passed into the continuous flow centrifuge by a gradient or/and pumps.

6. Process as claimed in one of the previous claims,
wherein
a continuous flow centrifuge whose centrifuge container has a volume of at least 0.1-50 l is used.

7. Process as claimed in one of the previous claims,
wherein
a continuous flow centrifuge whose centrifuge container has a volume of 0.2-4 l is used.

8. Process as claimed in one of the previous claims,
wherein
the size of the extrachromosomal DNA is 1 kbp-200 kbp.

9. Process as claimed in one of the previous claims,
wherein
the extrachromosomal DNA is linear, circular or supercoiled plasmid DNA.

10. Process as claimed in one of the previous claims,
wherein
the solution containing purified extrachromosomal DNA can be further purified.

11. Process as claimed in claim 10,
wherein
a further purification comprises an anion exchange chromatography, an affinity chromatography, a hydroxylapatite chromatography, an RNase treatment or/and a cross-flow filtration.

12. Process as claimed in one of the previous claims,
wherein
an extrachromosomal DNA is isolated which has essentially no strand breaks.

13. Use of an extrachromosomal DNA purified according to one of the previous claims for cloning, for transformation, for transfection, for microinjection into cells, for use in methods of gene therapy or/and for the polymerase chain reaction (PCR).

14. Use of a continuous flow centrifuge to purify extrachromosomal DNA.
